(19) United States
(12) Patent Application Publication
(10) Pub. No.: US 2010/0048984 A1
(43) Pub. Date: Feb. 25, 2010

(54) Integrated Sleep Aid Control Center and Method Therefor

(76) Inventor: Troy G. Anderson, Buckeye, AZ (US)

Correspondence Address: QUARLES & BRADY LLP, RENAISSANCE ONE, TWO NORTH CENTRAL AVENUE, PHOENIX, AZ, 85004-2391, US

(21) Appl. No.: 12/195,748

(22) Filed: Aug. 21, 2008

(51) Int. Cl. A61M 21/00

(52) U.S. Cl. 600/27; 600/26

(57) ABSTRACT

An integrated sleep control center has an enclosure and microcontroller disposed within the enclosure. A memory circuit is coupled to the microcontroller for storing programming data. An electronic interface is coupled to the microcontroller. A display shows status of the sleep control center. The sleep control center enclosure also contains LEDs to show status. A control knob sets programming attributes of the sleep control center. The electronic interface controls an environmental system which establishes a sleep environment. The environmental system can be lighting or temperature. The lighting has a wavelength of 400 nanometers. In addition, the environmental system can be audio/video equipment, olfactory, bio monitors, environmental systems, bed control, or appliances. The environmental system is controlled by electrical connection or wireless link from the sleep control center. The system is programmable to set environmental conditions which are conducive to initiating sleep, maintaining sleep, and minimizing stress when waking up.

### FIELD OF THE INVENTION

The present invention relates in general to electronic control systems and, more particularly, to an integrated sleep aid control center which controls a sleep environment.

### BACKGROUND OF THE INVENTION

Humans require a regular sleep pattern for health, well-being, and maximum effectiveness in daily activities. Yet, sleep disorders are a major medical issue for many people. Some of the health issues include seasonal affect disorder (SAD), delayed sleep phase syndrome (DSPS), bipolar disorders, and other circadian rhythm disorders. Failure to get regular sleep leads to lack of concentration, irregularity, eating disorders, fatigue, disorientation, insomnia, and degeneration of the immune system. The medical community distributes countless medications and other aids to help people fall asleep and obtain the rest needed by the body to rebuild from the daily activities.

For most people, the process of falling asleep and maintaining a restful state of sleep for the time required by the body to rejuvenate involves a variety of rituals and sleep inducing aids. Some people read, watch television, or otherwise wind down or relax prior to retiring for the evening. Others rely on over-the-counter or prescription medications to induce sleep. A typical pattern to prepare the mind and body for sleep may involve organizing things needed the next day, assisting children into bed, changing into bed attire, attending to facial and dental hygiene, participating in some form of relaxation, setting the alarm clock, and then laying in bed to await for sleep to begin. Yet, for many sleep may not come for a considerable period of time, potentially hours. Often the problem builds on itself as the failure to fall asleep, knowing that sleep is necessary, causes the mind and body to resist sleep.

The lack of regular sleep may be attributed to pressures of life, irregular times for initiating sleep, health issues, improper sleeping environment, or otherwise distracting mental thoughts. People often discount the importance of maintaining natural sleep patterns, yet become frustrated when sleep becomes problematic. In any case, without regular sleep, people suffer mentally and physically during the following day.

In the awakening process, conventional alarm clocks create buzzing, chimes, music, or other sounds with sufficient volume to awaken the person, typically with a sudden change of state. For many, the process of waking up using conventional methods, i.e., being rapidly brought from a sleep state to a conscious state by an abrupt, noisy alarm, imposes significant stress on the body. The person may exhibit an increase in heart rate and blood pressure, which is counterproductive to the purpose of sleep. People often rely on artificial stimulants, such as coffee, to fully wake up and prepare for the day ahead. The entire process can be unnatural and harmful to general health and well-being.

### SUMMARY OF THE INVENTION

A need exists to induce and maintain regular sleep patterns for health, well-being, and maximum mental and physical effectiveness, and awaken without inducing stress on the body. Accordingly, in one embodiment, the present invention is a sleep control center comprising an enclosure for housing electronic circuits. A display shows status of the sleep control center. A control knob sets programming attributes of the sleep control center. The sleep control center is adapted for interfacing to an environmental system which establishes a sleep-related environment.

In another embodiment, the present invention is an environmental sleep aid control system comprising an enclosure for housing electronic circuits. A display shows status of the sleep control center. A control knob sets programming attributes of the sleep control center. An environmental system is responsive to the electronic circuits to establish a sleep environment.

In another embodiment, the present invention is an integrated sleep control center comprising an enclosure and microcontroller disposed within the enclosure. A memory circuit is coupled to the microcontroller for storing programming data. An electronic interface is coupled to the microcontroller. A display shows the status of the sleep control center. A control knob sets programming attributes of the sleep control center. The electronic interface is adapted for controlling an environmental system which establishes a sleep environment.

In another embodiment, the present invention is a method of controlling a sleep environment comprising the steps of programming a sleep control center with selected timing and functions for an environmental system, and controlling the environmental system through the sleep control center to establish an environment conducive to sleep, maintaining sleep, and minimizing stress when waking up.

### BRIEF DESCRIPTION OF THE DRAWINGS

FIG. 1 is an integrated sleep control center with an electronic interface to environmental systems;

FIG. 2 is a block diagram of the various environmental systems which interface with the sleep control center; and

FIG. 3 is a block diagram of the electronic circuits within the sleep control center.

### DETAILED DESCRIPTION OF THE DRAWINGS

The present invention is described in one or more embodiments in the following description with reference to the Figures, in which like numerals represent the same or similar elements. While the invention is described in terms of the best mode for achieving the invention's objectives, it will be appreciated by those skilled in the art that it is intended to cover alternatives, modifications, and equivalents as may be included within the spirit and scope of the invention as defined by the appended claims and their equivalents as supported by the following disclosure and drawings.

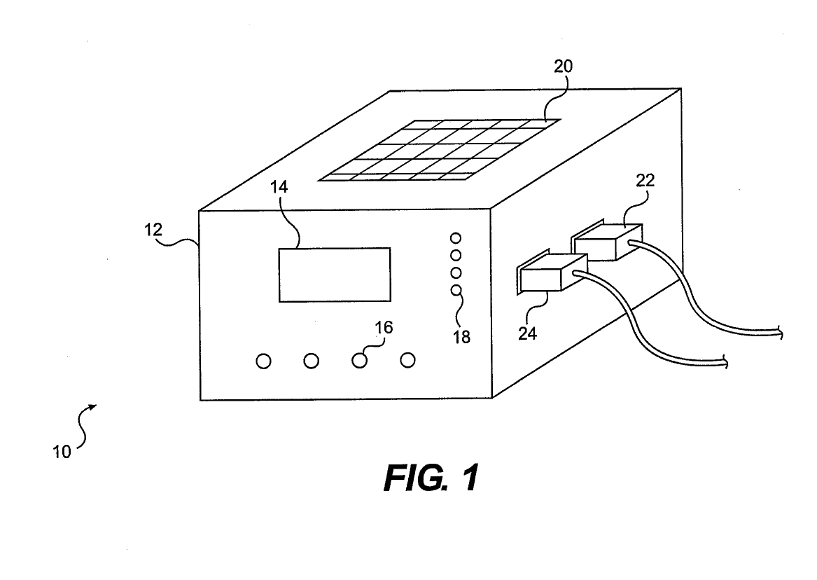

Turning to FIG. 1, an integrated sleep control center 10 is shown for creating a sleep-friendly environment. Sleep control center 10 is a mechanical and electronic device for controlling the physical properties of the sleep area and activities associated with initiating sleep and arising from the sleeping state. Sleep control center 10 typically resides near the bed for ready access and user operation.

Sleep control center 10 includes an enclosure 12 for housing electronic circuits which interface with environmental systems. Display 14 shows the status, programming attributes, and selected features of sleep control center 10, such as time, temperature, and environment system(s) enabled. Light indicators 18 provide additional status and selected features of sleep control center 10. Light indicators 18 can be light emitting diodes (LEDs). Control knobs 16 provide user control of the unit. A speaker 20 is provided in enclosure 12 for audio. Connectors 22 and 24 electrically connect to environmental systems which establish a sleep-related environment.

Sleep is one aspect of our daily circadian rhythm. In a 24-hour cycle, the body undergoes biochemical, physiological, and behavioral cycles. Circadian rhythms are endogenously generated, and can be entrained by environmental cues or influences, called Zeitgebers. Light is one example of Zeitgebers. These rhythms allow humans to anticipate and prepare for precise and regular environmental changes. Three general criteria of circadian rhythms are necessary to differentiate genuinely endogenous rhythms from coincidental or apparent ones: the rhythms persist in the absence of cues, they can be brought to match the local time, and do so in a precise manner over a range of temperatures.

True circadian rhythms persist in constant conditions, for example constant dark, with a period of about 24 hours. Circadian rhythms differ from apparent rhythms which merely are responses to external periodic cues. A rhythm cannot be endogenous unless it has been tested in conditions without external periodic input. The circadian rhythm is temperature-compensated, i.e., it maintains the same period over a range of temperatures. At a low enough or high enough temperature, the period of a circular reaction may reach 24 hours, but it will be merely coincidental. The apparent rhythm can be reset by exposure to an external stimulus. Circadian rhythms also differ from other imaginable endogenous 24-hour rhythms that are immune to resetting by external cues and hence do not serve the purpose of estimating the local time. Travel across time zones illustrates the necessity of the ability to adjust the biological clock so that it can reflect the local time and anticipate what will happen next.

Circadian rhythms are important in determining the sleeping and feeding patterns in humans. There are clear patterns of core body temperature, brain wave activity, hormone production, cell regeneration, and other biological activities linked to this daily cycle. In addition, photoperiodism, the physiological reaction of organisms to the length of day or night, is vital to humans, and the circadian system plays a role in the measurement and interpretation of daylength.

Circadian rhythms are linked to the light-dark cycle. People kept in total darkness for extended periods eventually function with a freerunning rhythm. Each day their sleep cycle is pushed back or forward, depending on whether their endogenous period is shorter or longer than 24 hours. Freerunning organisms that normally have one consolidated sleep episode will still have it when in an environment shielded from external cues, but the rhythm is not entrained to the 24-hour light/dark cycle in nature. The sleep/wake rhythm may, in these circumstances, become out of phase with other circadian or ultradian rhythms such as temperature and digestion.

The primary circadian clock in humans is located in the suprachiasmatic nucleus or nuclei (SCN), a pair of distinct groups of cells located in the hypothalamus. Irregularity or destruction of the SCN results in the absence of a regular sleep/wake rhythm. The SCN receives information about illumination through the eyes. The retina of the eyes contains not only classical photoreceptors, but also photoresponsive retinal ganglion cells. These cells, which contain a photo pigment called melanopsin, follow a pathway called the retinohypothalamic tract, leading to the SCN. If cells from the SCN are removed and cultured, they maintain their own rhythm in the absence of external cues. The SCN takes the information on day length from the retina, interprets it, and passes it on to the pineal gland located on the epithalamus. In response, the pineal gland secretes the hormone malatonin. Secretion of melatonin peaks at night and ebbs during the day.

Circadian rhythms are found in many organs and cells in the body outside the SCN. The SCN master clocks, called peripheral oscillators, are found in the esophagus, lung, liver, pancreas, spleen, thymus, and skin. The olfactory bulb and prostate may experience oscillations, suggesting that also these structures may be weak oscillators. Furthermore, liver cells, for example, appear to respond to feeding rather than to light. Cells from many parts of the body appear to have freerunning rhythms.

Light resets the biological clock in accordance with the phase response curve (PRC). Depending on the timing, light can advance or delay the circadian rhythm. Both the PRC and the required illuminance vary from species to species; much lower light levels are required to reset the clocks in nocturnal rodents than in humans. In addition to light intensity, wavelength (or color) of light is an important factor in the degree to which the clock is reset. Blue light in particular influences the circadian rhythm.

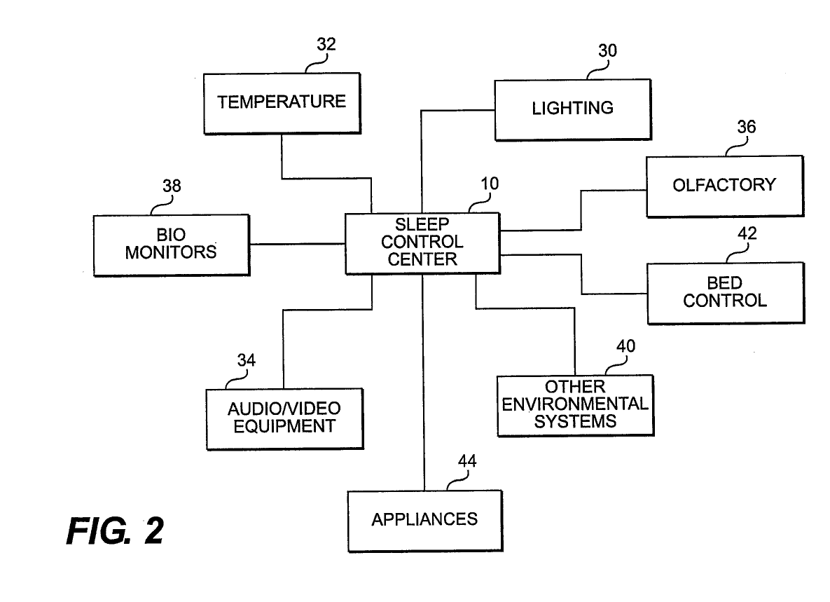

FIG. 2 is a block diagram showing integrated sleep control center 10 controlling a variety of environmental systems. Sleep control center 10 establishes an environment to aid with inducing sleep, maintaining continuous and restful sleep through the night, and waking up in the morning without subjecting the body to stress. By programming sleep control center 10 to set the conditions of the sleep environment, the circadian rhythms of the user or users can follow their natural pattern resulting in rapid lapse into the sleep state, quality rest, and minimal stress in the awakening process.

Sleep control center 10 is programmable using control knob 16 and display 14 to control the timing and function of the environmental systems. For example, sleep control center 10 controls lighting 30 by turning the light on and off, and setting the intensity and wavelength of light emitted. In one embodiment, lighting 30 is a lamp plugged into connector 22 or 24. A 400 nanometer (nm) wavelength light source is installed in the lamp. The 400 nm light source helps maintain the proper sleep environment in accordance with the user circadian rhythms. Sleep control center 10 activates lighting 30 via connector 22 or 24 at the selected time, e.g., 9 p.m. to begin the sleep inducing process. Sleep control center 10 turns off lighting 30 at the selected time, e.g., 11 p.m. Sleep control center 10 further turns lighting 30 back on in the morning at the selected time, e.g. 7 a.m., with the desired wavelength to initiate the awakening process. Thus, the sleeping person uses light to activate their awake cycle naturally, without the stress of abrupt and excessive audible sounds. As a backup, sleep control center 10 includes an audible alarm system via speaker 20 to ensure wakefulness in the event natural light inducing arousal fails to bring the person out of the sleeping state.

Alternatively, sleep control center 10 controls the entire lighting system in the bedroom or throughout the home to create the desired wavelength at the selected times in the environmental environment. The electronic circuits in sleep control center 10 are electrically connected to the home lighting system to control its operation. The user programs sleep control center 10 to active lighting 30 at the selected time and selected wavelength and intensity, and maintain the light for the desired durations of time. By controlling lighting 30, sleep control center 10 creates an environment to aid with inducing sleep, maintaining continuous and restful sleep through the night, and waking up in the morning without subjecting the body to stress. The user awakes rested without the stress or increase in blood pressure or heart rate attributed to conventional alarm clocks.

Sleep control center 10 also controls temperature 32 according to the user programming, which sets and regulates the temperature of the environment during the sleep cycle. The electronic circuits in sleep control center 10 are electrically connected to the home heating and cooling system to regulate temperature. The temperature can vary during the night, e.g., between 69-72° F., as per the user's programming instructions.

Sleep control center 10 can activate audio/video equipment 34 according to the user programming. The electronic circuits in sleep control center 10 are electrically connected to audio/video equipment 34 to control its operation. The audio/video equipment provides sounds and visual images to induce sleep, maintain sleep, and minimize stress when waking up. Sleep control center 10 can activate olfactory block 36 to provide smells to induce sleep, maintain sleep, and minimize stress when waking up. For example, olfactory block 36 may introduce a floral scent or ocean sounds to induce sleep, and a coffee or breakfast aroma to help the person wake up.

Sleep control center 10 controls bio monitors 38 which connect to sensors placed on the sleeping person to monitor bio functions such as heart rate, blood pressure, body temperature, and brain activity. The bio function can be integrated into sleep control center 10 to modify the programming. For example, if bio monitor senses that the sleeping person's body temperature is rising, then the environmental temperature 32 can be decreased. If bio functions register excessive stress during the awakening process, then the awakening cues, i.e., light intensity, can be reduced.

Sleep control center 10 further controls other environmental systems, such as humidity, air circulation, and opening or closing blinds on a window. The attributes of the bed can be controlled in block 42. For example, the head of the bed may be raised in the morning to a sitting position for the sleeping person during the awakening process. The firmness of the mattress can be adjusted by sleep control center 10. Finally, sleep control center 10 can control appliances 44 such as coffee maker, microwave oven, or toaster. The environmental systems 30-44 are all part of the sleep-related environment.

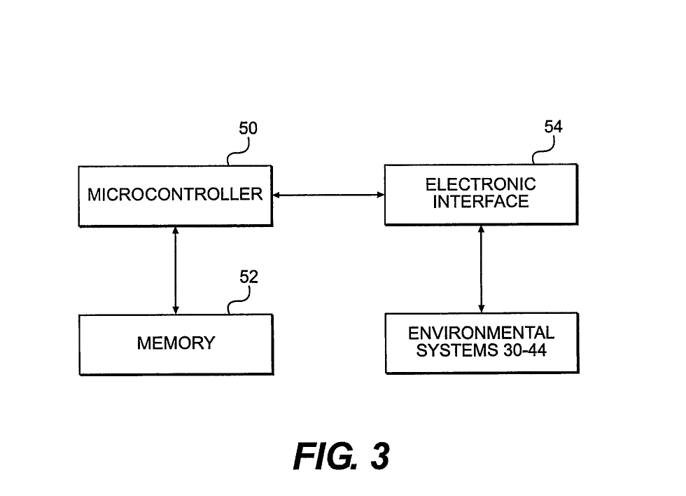

Further detail of the electronic circuit in sleep control center 10 is shown in FIG. 3. Microcontroller 50 executes the programming instructions selected by the user. Memory 52 is coupled to microcontroller 50 and stores the programming data. The user selects the operating conditions of the environmental system with control knobs 16. For example, lighting 30 can be set to turn on and off at selected times. Electronic interface 54 receives instructions from microcontroller 50 at the designated times and controls environmental systems 30-44. Electronic interface 54 contains electronic drivers which can be hard-wired through connectors 22 and 24 or transmitted by wireless radio frequency (RF) link to environmental systems 30-44. The programming feature is implemented through control knobs 16 and stored in memory 52.

Sleep education and counseling information on sleep hygiene and other sleep issues from medical professionals is stored in memory 52 or compact disk (CD) which can be played on sleep control center 10. Similar information is available in documentation provided with sleep control center 10 or an associated internet website.

In summary, the sleep control center allows the user to program the environmental conditions which are most conducive to sleep, maintaining sleep, and minimizing stress when waking up. The sleep control center executes the programming instructions to set the environmental conditions to match the circadian rhythms of the user.

While one or more embodiments of the present invention have been illustrated in detail, the skilled artisan will appreciate that modifications and adaptations to those embodiments may be made without departing from the scope of the present invention as set forth in the following claims.

What is claimed:

1. A sleep control center, comprising:
an enclosure for housing electronic circuits;
a display for displaying status of the sleep control center; and
a control knob for setting programming attributes of the sleep control center, wherein the sleep control center is adapted for interfacing to an environmental system which establishes a sleep environment.

2. The sleep control center of claim 1, wherein the environmental system is lighting or temperature.

3. The sleep control center of claim 2, wherein the lighting has a wavelength of 400 nanometers.

4. The sleep control center of claim 1, wherein the environmental system is audio/video equipment, olfactory, bio monitors, environmental systems, bed control, or appliances.

5. The sleep control center of claim 1, further including:
a microcontroller disposed within the enclosure;
a memory circuit coupled to the microcontroller for storing programming data; and
an electronic interface coupled to the microcontroller for controlling the environmental system.

6. The sleep control center of claim 5, wherein the electronic interface includes an electrical connector or wireless link.

7. The sleep control center of claim 1, wherein the system is programmable to set environmental conditions which are conducive to initiating sleep, maintaining sleep, and minimizing stress when waking up.

8. An environmental sleep aid control system, comprising:
an enclosure for housing electronic circuits;
a display for displaying status of the sleep control center;
a control knob for setting programming attributes of the sleep control center; and
an environmental system responsive to the electronic circuits to establish a sleep environment.

9. The environmental sleep aid system of claim 8, wherein the environmental system is lighting or temperature.

10. The environmental sleep aid system of claim 9, wherein the lighting has a wavelength of 400 nanometers.

11. The environmental sleep aid system of claim 8, wherein the environmental system is audio/video equipment, olfactory, bio monitors, environmental systems, bed control, or appliances.

12. The environmental sleep aid system of claim 8, further including:
a microcontroller disposed within the enclosure;
a memory circuit coupled to the microcontroller for storing programming data; and
an electronic interface coupled to the microcontroller for controlling the environmental system.

13. The environmental sleep aid system of claim 12, wherein the electronic interface includes an electrical connector or wireless link.

14. An integrated sleep control center, comprising:
an enclosure;
a microcontroller disposed within the enclosure;
a memory circuit coupled to the microcontroller for storing programming data;
an electronic interface coupled to the microcontroller;
a display for displaying status of the sleep control center; and
a control knob for setting programming attributes of the sleep control center, wherein the electronic interface is adapted for controlling an environmental system which establishes a sleep environment.

15. The sleep control center of claim 14, wherein the environmental system is lighting or temperature.

16. The sleep control center of claim 15, wherein the lighting has a wavelength of 400 nanometers.

17. The sleep control center of claim 14, wherein the environmental system is audio/video equipment, olfactory, bio monitors, environmental systems, bed control, or appliances.

18. The sleep control center of claim 14, wherein the electronic interface includes an electrical connector or wireless link.

19. The sleep control center of claim 14, wherein the system is programmable to set environmental conditions which are conducive to initiating sleep, maintaining sleep, and minimizing stress when waking up.

20. A method of controlling a sleep environment, comprising:
programming a sleep control center with selected timing and functions for an environmental system; and
controlling the environmental system through the sleep control center to establish an environment conducive to initiating sleep, maintaining sleep, and minimizing stress when waking up.

21. The method of claim 20, wherein the environmental system is lighting or temperature.

22. The method of claim 21, wherein the lighting has a wavelength of 400 nanometers.

23. The method of claim 20, wherein the environmental system is audio/video equipment, olfactory, bio monitors, environmental systems, bed control, or appliances.

24. The method of claim 20, wherein the environmental system is controlled by electrical connector or wireless link.
